(19) United States
(12) Patent Application Publication
(10) Pub. No.: US 2009/0299171 A1
(43) Pub. Date: Dec. 3, 2009

(54) Intraluminal Access and Imaging Device

(75) Inventors: Niall Duffy, Ballybrit (IE); Sean Whelan, Ballybrit (IE)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., SANTA ROSA, CA (US)

(21) Appl. No.: 12/132,411

(22) Filed: Jun. 3, 2008

(51) Int. Cl. A61B 5/05; A61B 6/00; A61M 31/00

(52) U.S. Cl. 600/420; 600/424; 600/433; 604/508

(57) ABSTRACT

An intraluminal access and imaging device is configured to image a path of a vessel that includes a chronic total occlusion. The device includes an elongated member having a distal end and a proximal end. The distal end is configured to pass through an inner layer of the vessel so that the elongated member may track subintimally along a side of the chronic total occlusion. The elongated member includes a radiopaque material configured to be highly visible under fluoroscopy.

### BACKGROUND OF THE INVENTION

1. Field of the Invention

The present invention is generally related to an intraluminal access and imaging device.

2. Background of the Invention

Stenotic lesions may comprise a hard, calcified substance and/or a softer thrombus material, each of which forms on the lumen walls of a blood vessel and restricts blood flow there through. Intra-luminal treatments such as balloon angioplasty (PTA, PTCA, etc.), stent deployment, atherectomy, and thrombectomy are well known and have proven effective in the treatment of such stenotic lesions. These treatments often involve the insertion of a therapy catheter into a patient's vasculature, which may be tortuous and may have numerous stenoses of varying degrees throughout its length. In order to place the distal end of a catheter at the treatment site, a guidewire is typically introduced and tracked from an incision, through the vasculature, and across the lesion. Then, a catheter (e.g. a balloon catheter), perhaps containing a stent at its distal end, can be tracked over the guidewire to the treatment site. Ordinarily, the distal end of the guidewire is quite flexible so that it can be rotatably steered and pushed through the bifurcations and turns of the typically irregular passageway without damaging the vessel walls.

In some instances, the extent of occlusion of the lumen is so severe that the lumen is completely or nearly completely obstructed, which may be described as a total occlusion. If this occlusion persists for a long period of time, the lesion is referred to as a chronic total occlusion or CTO. Furthermore, in the case of diseased blood vessels, the lining of the vessels may be characterized by the prevalence of atheromatous plaque, which may form total occlusions. The extensive plaque formation of a chronic total occlusion typically has a fibrous cap surrounding softer plaque material. This fibrous cap may present a surface that is difficult to penetrate with a conventional guidewire, and the typically flexible distal tip of the guidewire may be unable to cross the lesion.

Thus, for treatment of total occlusions, stiffer guidewires have been employed to recanalize through the total occlusion. However, due to the fibrous cap of the total occlusion, a stiffer guidewire still may not be able to cross the occlusion.

Further, in a CTO, there may be a distortion of the regular vascular architecture such that there may be multiple small non-functional channels throughout the occlusion rather than one central lumen for recanalization. Thus, the conventional approach of looking for the single channel in the center of the occlusion may account for many of the failures. Furthermore, these spontaneously recanalized channels may be responsible for failures due to their dead-end pathways and misdirecting of the guidewires. Once a “false” tract is created by a guidewire, subsequent attempts with different guidewires may continue to follow the same incorrect path, and it is very difficult to steer subsequent guidewires away from the false tract.

Another equally important failure mode, even after a guidewire successfully crosses a chronic total occlusion, is the inability to advance a balloon or other angioplasty equipment over the guidewire due to the fibrocalcific composition of the chronic total occlusion, mainly both at the “entry” point and at the “exit” segment of the chronic total occlusion. Even with balloon inflations throughout the occlusion, many times there is no antegrade flow of contrast injected, possibly due to the recoil or insufficient channel creation throughout the occlusion.

Successful recanalization of chronic total occlusions remains an area where improvements are needed. Approximately 30% of all coronary angiograms in patients with coronary artery disease will show a CTO and its presence often excludes patients from treatment by percutaneous coronary intervention. Acute success rates vary according to the duration of occlusion, the morphology of the lesion and the coronary anatomy, the experience of the clinician, the degree of persistence employed, and the type of equipment used. Recanalization rates range between 45-80%, with the highest success in short, recently occluded (<1 month), non-calcified lesions.

It is desirable to be able to visualize the CTO in terms of its length and shape before attempting to cross the CTO with a guidewire to minimize potential trauma to the area of the lumen at or near the CTO. Having the ability to visualize the CTO prior to attempting to cross or otherwise bypass the CTO may increase the chance of success of crossing the CTO with a guidewire without damaging surrounding tissue, or bypassing the CTO using other known methods.

### SUMMARY OF THE INVENTION

The present invention describes an apparatus and method to access the intermediate layer of a vessel adjacent a CTO and provide imaging to assist a clinician to cross or bypass the CTO.

According to an aspect of the present invention, there is provided an intraluminal access and imaging device for imaging a path of a vessel comprising a chronic total occlusion. The device includes an elongated member having a distal end and a proximal end. The distal end is configured to pass through an inner layer of the vessel so that the elongated member may track subintimally along a side of the chronic total occlusion. The elongated member includes a radiopaque material configured to be highly visible under fluoroscopy.

According to an aspect of the invention, there is provided an intraluminal access and imaging device for imaging a path of a vessel comprising a chronic total occlusion. The device includes an elongated member having a distal end and a proximal end. The distal end is configured to pass through an inner wall of the vessel so that the elongated member may track in an intermediate layer of the vessel along a side of the chronic total occlusion. The elongated member includes a coil configured to be visible under external magnetic imaging.

According to an aspect of the invention, there is provided an intraluminal access and imaging device for imaging a path of a vessel comprising a chronic total occlusion. The device includes an elongated member having a distal end and a proximal end. The distal end is configured to pass through an inner wall of the vessel so that the elongated member may track in an intermediate layer of the vessel along a side of the chronic total occlusion. The elongated member includes a plurality of ports configured to deliver a substance to the intermediate layer.

According to an aspect of the invention, there is provided a method for visualizing a path of a chronic total occlusion. The method includes inserting an elongated member that includes a radiopaque material into a lumen of a vessel comprising the chronic total occlusion, passing through an inner wall of the vessel with a distal end of the elongated member, tracking the elongated member subintimally along side the chronic total occlusion, and monitoring the elongated member with an external imaging system configured to image the radiopaque material.

According to an aspect of the invention, there is provided a method for treating a chronic total occlusion. The method includes inserting an elongated member comprising a plurality of micro ports into a lumen of a vessel comprising the chronic total occlusion, passing through an inner layer of the vessel with a distal end of the elongated member, tracking the elongated member subintimally along side the chronic total occlusion, and injecting a therapeutic agent into an intermediate layer of the vessel through the plurality of micro ports.

According to an aspect of the invention, there is provided a method for imaging a chronic total occlusion. The method includes inserting an elongated member that includes a coil into a lumen of a vessel that includes the chronic total occlusion. The method also includes passing through an inner layer of the vessel with a distal end of the elongated member, tracking the elongated member subintimally along side the chronic total occlusion, and monitoring the position of the elongated member with an external magnetic imaging system.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1 is a schematic diagram of a vessel with a chronic total occlusion;

FIG. 2 is a schematic diagram of an intraluminal access and imaging device according to an embodiment of the present invention;

FIG. 3 is a schematic diagram of a distal end of an elongated member of the intraluminal access and imaging device of FIG. 2;

FIG. 4 is a schematic diagram of an embodiment of the elongated member of the intraluminal access and imaging device of FIG. 2;

FIG. 5 is a schematic diagram of an embodiment of the elongated member of the intraluminal access and imaging device of FIG. 2;

FIG. 6 is a schematic diagram of an embodiment of the elongated member of the intraluminal access and imaging device of FIG. 2 being subintimally tracked in the vessel of FIG. 1 next to the CTO;

FIG. 7 is a schematic diagram of an embodiment of the elongated member of the intraluminal access and imaging device of FIG. 2;

FIG. 8 is a schematic diagram of an embodiment of the elongated member of the intraluminal access and imaging device of FIG. 2; and

FIG. 9 is a schematic diagram of an embodiment of the embodiment of the elongated member of FIG. 8 being subintimally tracked in the vessel of FIG. 1 next to the CTO.

### DETAILED DESCRIPTION OF THE INVENTION

The following detailed description is merely exemplary in nature and is not intended to limit the invention or the application and use of the invention. Furthermore, there is no intention to be bound by any expressed or implied theory presented in the preceding technical field, background, brief summary or the following detailed description.

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

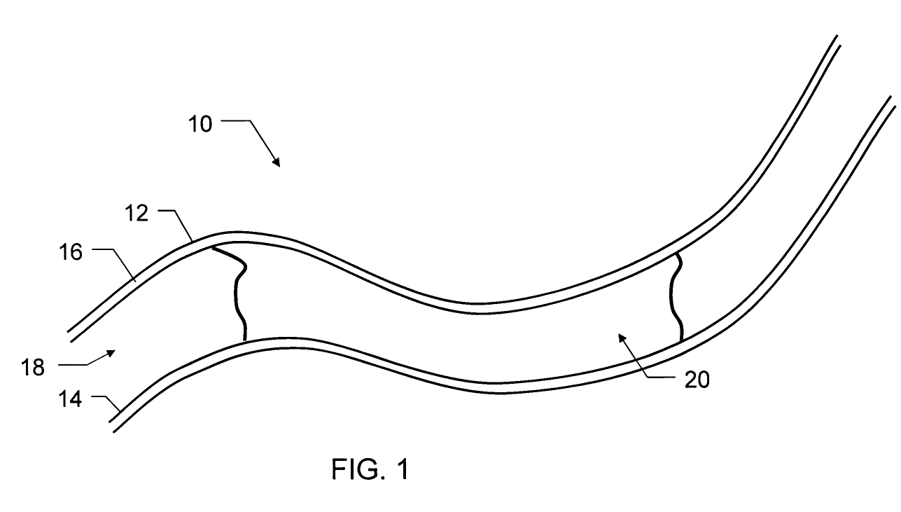

FIG. 1 illustrates a vessel 10 having an outer layer 12, also known as the tunic adventitia or adventitial layer, an inner layer 14, also known as the tunic intima or intimal layer, and an intermediate layer 16 in between the outer layer 12 and the inner layer 14. The intermediate layer 16 is also known as the tunic media, or medial layer. The inner layer 14 provides a wall that defines a lumen 18 through which blood flows. A chronic total occlusion 20 is located in the lumen 18 and substantially blocks or totally blocks the blood flow through the lumen 18.

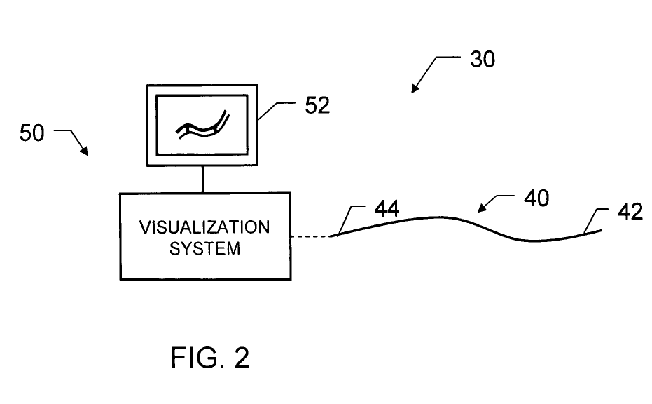

FIG. 2 illustrates an intraluminal access and imaging device 30 according to an embodiment of the present invention. As illustrated, the intraluminal access and imaging device 30 includes an elongated member 40, which may be in the form of a wire, or any other suitable flexible material. The elongated member 40 has a distal end 42 that is configured to enter the lumen 18 and pass through the inner layer 14 of the vessel in a controlled manner so that the elongated member 40 may track the CTO 20 in the intermediate layer 16, as discussed in further detail below. The elongated member 40 also has a proximal end 44 that is configured to stay outside of the patient and be handled by the clinician. The distal end 42 is relatively rigid, especially as compared to conventional guidewires, which tend to have relatively soft tips.

At least the distal end 42 of the elongated member 40 includes or is made from a radiopaque material. In an embodiment, the entire elongated member 40 is made from a highly radiopaque material, which enables the elongated member 40 to be visible in a radiographic image, such as an x-ray or fluoroscopic image. Radiopaque materials do not allow certain wavelengths of radiation, such as x-rays, to pass through, which allows the clinician to see the radiopaque material in a human body when using suitable visualization equipment, such as a fluoroscope.

Any suitable radiopaque material that allows the elongated member 40 to be imaged with imaging equipment, while allowing the elongated member to maintain flexibility, may be used. Examples of such radiopaque materials include, but are not limited to metals such as gold, platinum, and alloys thereof, and filled polymeric materials, such as barium sulfate loaded silicone, polyimide, and polycarbonate. The distal end 42 of the elongated member 40 may be made of a material that is radiopaque, but is more rigid than the remaining portion of the elongated member 40 so that it may pass through the inner layer 14 without distorting.

The intraluminal access and imaging device 30 also includes a visualization system 50. The visualization system 50 is constructed and arranged to allow the vessel 10 to be visualized. For example, in an embodiment, the visualization system is constructed and arranged to detect radiopaque material. Such visualization equipment is known in the art and therefore is not discussed in greater detail herein. For example, the visualization system 50 may be configured to use X-ray fluoroscopy and may include a fluoroscope, which is commonly used in surgical procedures. As discussed in further detail below, in an embodiment, the visualization system 50 may be constructed and arranged to use external magnetic imaging, such as magnetic resonance imaging (“MRI”) to allow the vessel 10 to be visualized.

The visualization system 50 also includes an output device 52 that is configured to output an image of the vessel 10 that is created by the visualization system 50. In an embodiment, the output device 52 is a monitor, as illustrated in FIG. 2. The clinician operating the intraluminal access and imaging device 30 may use the image to manipulate the elongated member 40 in the vessel 10.

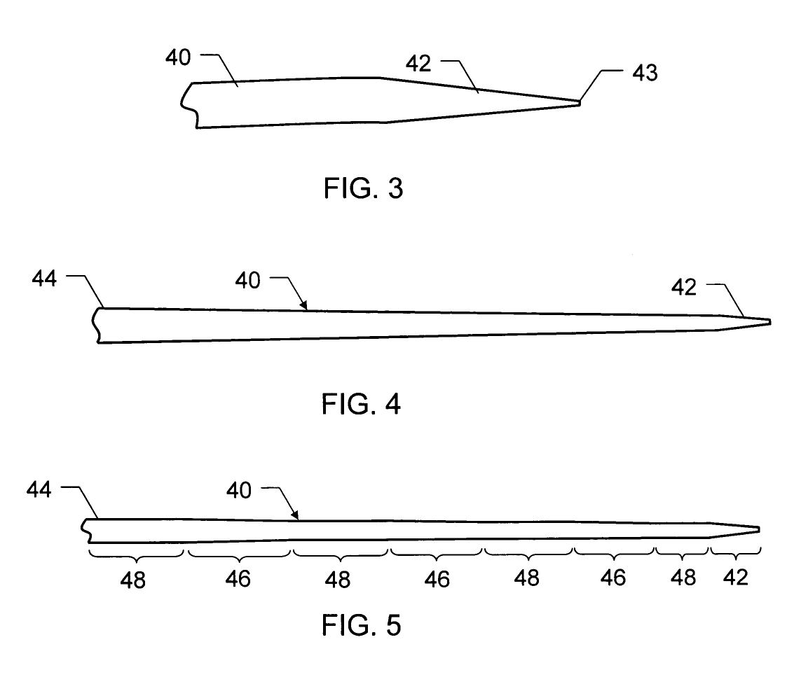

FIG. 3 illustrates an embodiment of the elongated member 40. As illustrated, the distal end 42 of the elongated member 40 is tapered so that a distal tip 43 of the elongated member 40 has a diameter that is smaller than the diameter of the remaining portions of the elongated member 40. The distal end 42 is rigid and is configured to pass through the inner layer 14 of the vessel in a controlled manner when the elongated member 40 is steered towards the inner layer 14 at a suitable angle. The distal end 42 differs from conventional distal ends of guidewires that are designed to be soft and not cause trauma to the vessel wall. Once the distal end 42 has passed through the inner layer 14 of the vessel 10, it may track the vessel 10 subintimally, i.e., in the intermediate layer 16. It is desirable to control the distal end 42 of the elongated member 40 so that the distal end 42 does not pass through the outer layer 12, but instead stays within the intermediate layer 16.

In an embodiment, illustrated in FIG. 4, the elongated member 40 has a substantially constant taper along its length. The constant taper may allow for a small diameter at the distal tip 43 to pass through the inner layer 14, and provide a more substantial diameter proximal to the distal tip 43 to allow for sufficient radiopacity.

FIG. 5 illustrates an embodiment in which the elongated member 40 includes a plurality of tapered sections 46 along its length. A plurality of substantially straight sections 48 are also provided so that two tapered sections 46 are separated by a straight section 48, as illustrated. Similar to the embodiment of FIG. 4, the use of tapered sections 46 may allow for the smaller diameter at the distal tip 43 to pass through the inner layer 14, and a more substantial proximal diameter for sufficient radiopacity.

The illustrated embodiments of the elongated member 40 are not intended to be limiting in any way, and any suitable shape that allows the distal tip 43 to pass through the inner layer 14 and also allows the elongated member 40 to have sufficient radiopacity may be used. The elongated member 40 may be a single piece of material that has been formed into any of the shapes illustrated in FIGS. 3-5. In an embodiment, the elongated member 40 may include a plurality of sections that are individually shaped and connected together via any suitable technique, such as welding or soldering.

In operation, the elongated member 40 is tracked in the lumen 18 of the vessel 10 to the CTO 20. The elongated member 40 may be tracked to the CTO 20 on its own, or may be tracked through a guide catheter that has been tracked to a position just proximal to the CTO 20 with a conventional guidewire. In embodiments where a guide catheter is used, once the guide catheter has been tracked to the desired position relative to the CTO, e.g., just proximal to the CTO, the guidewire may be withdrawn from the guide catheter, and the elongated member 40 may be front loaded into the guide catheter and tracked to the CTO 20. Once the elongated member 40 is located just proximal to the CTO 20, the guide catheter may be removed while keeping the elongated member 40 in place.

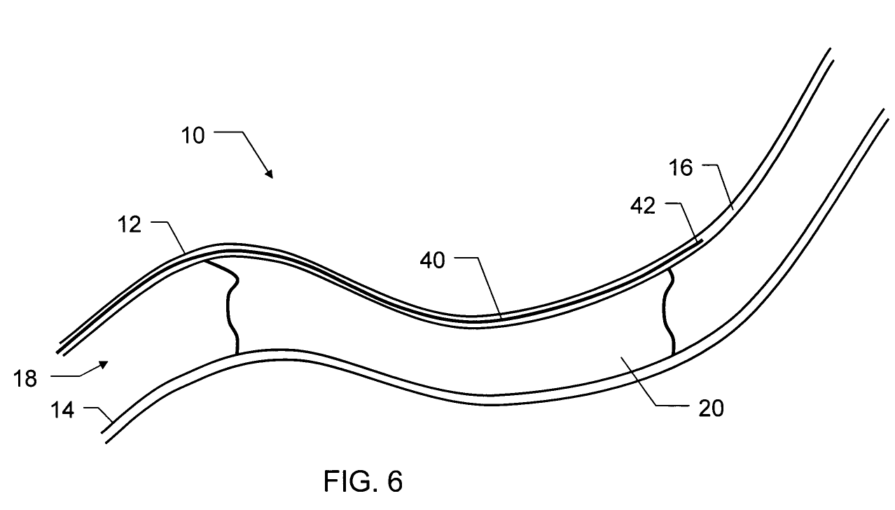

Because at least the distal end 42 of the elongated member 40 is radiopaque, it may be carefully steered through the inner layer 14 and into the intermediate layer 16, without piercing the outer layer 12 of the vessel 10. The elongated member 40 may then be tracked along the CTO 20 within the intermediate layer 16, as shown in FIG. 6. In an embodiment, the elongated member 40 may be used with an imaging catheter, such as the PIONEER catheter manufactured by Medtronic Vascular, Inc. (Santa Rosa, Calif.), that is configured to reenter the lumen 18 at a location distal of the CTO 20.

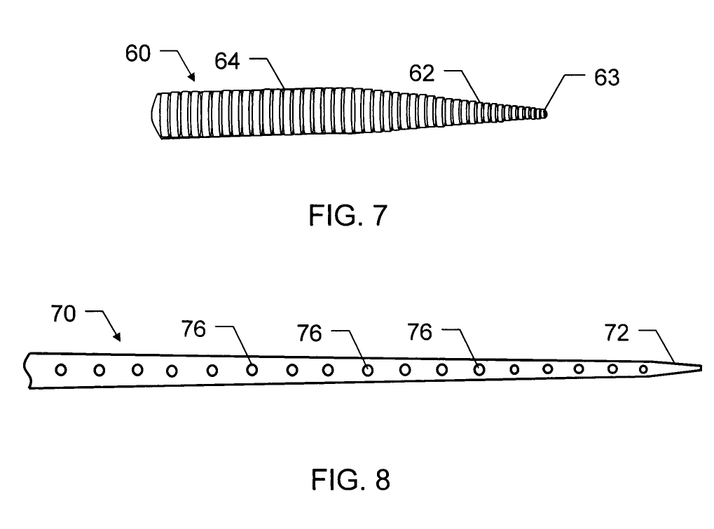

FIG. 7 illustrates an embodiment of an elongated member 60 that includes a rigid distal end 62 that is constructed and arranged to pass through the inner layer 14 of the vessel 10 so that the elongated member 60 may track the CTO 20 subintimally, i.e., in the intermediate layer 16. In the illustrated embodiment, the distal end 62 is tapered such that a distal tip 63 thereof has the smallest diameter of the elongated member 60, similar to the embodiment of the elongated member 40 described above.

As illustrated, the elongated member 60 also includes a coil 64. The coil 64 may be constructed and arranged so that the elongated member 60 is visible when using an external magnetic imaging system as the visualization system 50, such as an MRI system. Examples of such MRI systems include, but are not limited to systems from GE Medical Systems, Waukesha, Wis.; Siemens Medical Solutions of Siemens AG, Malvern, Pa. and Erlangen, Germany; and Toshiba America Medical Systems, Inc., Tustin, Calif. The visualization system 50 may be configured to detect the coil 64 of the elongated member 60 and convert the signal received from the coil 64 to an image outputted by the output device 52. This allows the clinician to visualize the path of the vessel 10 and CTO 20.

The coil 64 may be considered to be an MRI receiver coil that is configured to be detected by the visualization system 50 (in this embodiment an MRI system) and displayed on the output device 52. This enables the clinician to determine, from the MRI display, when the distal end 62 of the elongated member 60 has entered the intermediate layer 16, as well as follow movement of the distal end 62 as it tracks the CTO 20 subintimally.

The coil 64 may only be provided to the distal end 62 of the elongated member 60, or in an embodiment, the coil 64 may be provided to the entire length of the elongated member 60. In an embodiment, a plurality of coils may be provided so as to provide rings or bands of windings along the distal portion of the elongated member or along the entire length of the elongated member. The illustrated embodiment is not intended to be limiting in any way. For example, in an embodiment, different coils may be connected to different channels in the visualization system 50, such that each coil may be depicted on the MRI image in a different color to further enhance the visualization of the path to cross the CTO 20.

The coil 64 may be made of a conductive material that is shielded along the majority of its length to inhibit interference, as is known in the art. Although a coil is illustrated in FIG. 7, it should be understood that other devices to create an image in an MRI system may also be used, including objects which may not be literally a coil. The coil 64 is connected to a proximal connector located at the proximal end of the elongated member 60. The proximal connector may be connected to the visualization system 50 through an impedance matching circuit, as is known in the art, such that signals from the coil 64 are received by the visualization system 50. The visualization system 50 may be programmed to display, in response to such signals received from the coil 64, the position of the coil 64 relative to anatomical structures within the subject's body in a so-called real time manner. In an embodiment, the image generated by the visualization system 50 may be superimposed into a prior MRI imaging scan, or even an image created by fluoroscopy, stored in memory of the visualization system 50 using known techniques.

FIG. 8 illustrates an embodiment of an elongated member 70 that includes a rigid distal end 72 that is constructed and arranged to pass through the inner layer 14 of the vessel 10 so that the elongated member 70 may track the CTO 20 subintimally, as described in embodiments above. The elongated member 70 may include a highly radiopaque material or a coil, as described above, so that the clinician may use a suitable visualization system (e.g., a fluoroscope or an MRI system) to visualize the path of the vessel 10 and the CTO 20.

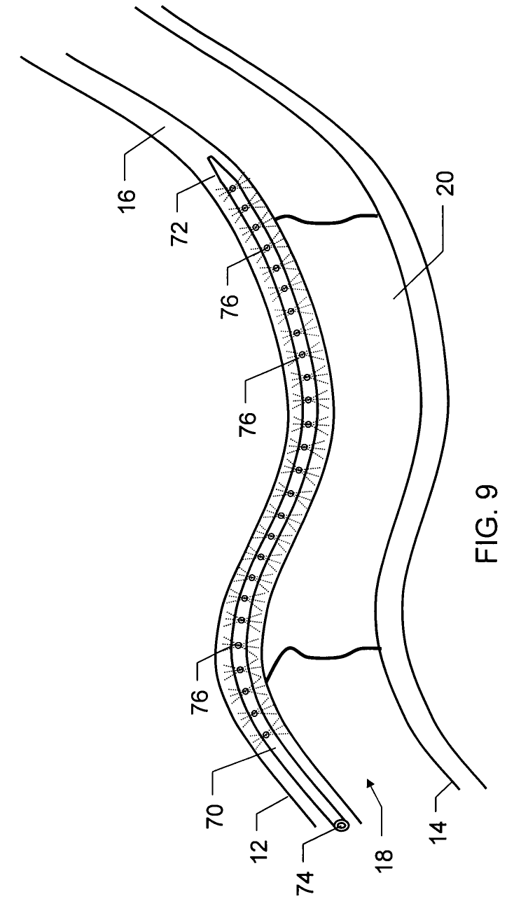

The elongated member 70 includes a passageway 74 (see FIG. 9) that is connected to a plurality of micro ports 76. As illustrated, the micro ports 76 are located along at least a portion of a length of the elongated member 70 extending proximal from the distal end 72. As shown in FIG. 9, the passageway 74 and the micro ports 76 are configured to deliver contrast, such as gadolinium or manganese, to the intermediate layer 16 to further enhance the image of the path of the vessel 10 when using the visualization system 50. By providing contrast to the intermediate layer 16, the intermediate layer 16 will become stained evenly along the side of the CTO 20. This may provide a way to visualize the path of the vessel 10 and the CTO 20 using minimum contrast, as compared to conventional methods that inject contrast into the lumen 18 of the vessel 10.

In an embodiment, the elongated member 70 may be used to deliver a therapeutic agent subintimally through the micro ports 76. Examples of therapeutic agents include but are not limited to agents that may soften the CTO 20 so that the CTO 20 may be easier to cross with a guidewire and/or catheter, and agents that may prevent restenosis. Any other suitable therapeutic agent may be delivered to the intermediate layer 16 through the micro ports 76.

While embodiments have been presented in the foregoing detailed description of the invention, it should be appreciated that a vast number of variations exist. It should also be appreciated that the embodiments described herein are only examples, and are not intended to limit the scope, applicability, or configuration of the invention in any way. Rather, the foregoing detailed description will provide those skilled in the art with a convenient roadmap for implementing an exemplary embodiment of the invention, it being understood that various changes may be made in the function and arrangement of elements described in an exemplary embodiment without departing from the scope of the invention as set forth in the appended claims.

What is claimed is:

1. An intraluminal access and imaging device for imaging a path of a vessel comprising a chronic total occlusion, the device comprising:
an elongated member having a distal end and a proximal end, the distal end being configured to pass through an inner layer of the vessel so that the elongated member may track subintimally along a side of the chronic total occlusion, the elongated member comprising a radiopaque material configured to be highly visible under fluoroscopy.

2. An intraluminal access and imaging device according to claim 1, wherein at least the distal end comprises the radiopaque material.

3. An intraluminal access and imaging device according to claim 2, wherein the entire elongated member comprises the radiopaque material.

4. An intraluminal access and imaging device according to claim 1, wherein the distal end is tapered.

5. An intraluminal access and imaging device according to claim 4, wherein the elongated member comprises a plurality of tapers, and wherein the proximal end has a greater diameter than the distal end.

6. An intraluminal access and imaging device according to claim 5, wherein the entire elongated member comprises the radiopaque material.

7. An intraluminal access and imaging device according to claim 1, wherein the elongated member is a wire.

8. An intraluminal access and imaging device according to claim 1, further comprising a visualization system constructed and arranged to output an image of at least the distal end of the elongated member.

9. An intraluminal access and imaging device according to claim 8, wherein the visualization system comprises a fluoroscope.

10. An intraluminal access and imaging device for imaging a path of a vessel comprising a chronic total occlusion, the device comprising:
an elongated member having a distal end and a proximal end, the distal end being configured to pass through an inner wall of the vessel so that the elongated member may track in an intermediate layer of the vessel along a side of the chronic total occlusion, the elongated member comprising a coil configured to be visible under external magnetic imaging.

11. An intraluminal access and imaging device according to claim 10, further comprising a visualization system constructed and arranged to output an image of at least the coil.

12. An intraluminal access and imaging device according to claim 11, wherein the visualization system comprises a magnetic resonance imaging system.

13. An intraluminal access and imaging device for imaging a path of a vessel comprising a chronic total occlusion, the device comprising:
an elongated member having a distal end and a proximal end, the distal end being configured to pass through an inner wall of the vessel so that the elongated member may track in an intermediate layer of the vessel along a side of the chronic total occlusion, the elongated member comprising a plurality of ports configured to deliver a substance to the intermediate layer.

14. An intraluminal access and imaging device according to claim 13, wherein the substance comprises a contrast.

15. An intraluminal access and imaging device according to claim 13, wherein the substance comprises a therapeutic agent.

16. A method for visualizing a path of a chronic total occlusion, the method comprising:
inserting an elongated member comprising a radiopaque material into a lumen of a vessel comprising the chronic total occlusion;
passing through an inner wall of the vessel with a distal end of the elongated member;
tracking the elongated member subintimally along side the chronic total occlusion; and
monitoring the position of the elongated member with an external imaging system configured to image the radiopaque material.

17. A method according to claim 16, further comprising injecting a contrast in an intermediate layer of the vessel via a plurality of micro ports in the elongated member.

18. A method for treating a chronic total occlusion, the method comprising:
inserting an elongated member comprising a plurality of micro ports into a lumen of a vessel comprising the chronic total occlusion;
passing through an inner layer of the vessel with a distal end of the elongated member;
tracking the elongated member subintimally along side the chronic total occlusion; and
injecting a therapeutic agent into an intermediate layer of the vessel through the plurality of micro ports.

19. A method for imaging a chronic total occlusion, the method comprising:
inserting an elongated member comprising a coil into a lumen of a vessel comprising the chronic total occlusion;
passing through an inner layer of the vessel with a distal end of the elongated member;
tracking the elongated member subintimally along side the chronic total occlusion; and
monitoring the position of the elongated member with an external magnetic imaging system.

20. A method for imaging a chronic total occlusion according to claim 19, further comprising superimposing an image provided by said tracking onto a fluoroscopic image of the vessel.
